(19) United States
(12) Patent Application Publication
(10) Pub. No.: US 2023/0335243 A1
(43) Pub. Date: Oct. 19, 2023

(54) PIPELINE FOR INTELLIGENT TEXT ANNOTATION OF MEDICAL REPORTS VIA ARTIFICIAL INTELLIGENCE BASED NATURAL LANGUAGE PROCESSING WORKFLOWS

(71) Applicant: GE Precision Healthcare LLC, Milwaukee, WI (US)

(72) Inventors: Amitabh Mukherjee, Bangalore (IN); M. S. Raghu Prasad, Bangalore (IN)

(21) Appl. No.: 17/659,562

(22) Filed: Apr. 18, 2022

(51) Int. Cl. G16H 15/00

(52) U.S. Cl. CPC G16H 15/00

(57) ABSTRACT

Techniques relating to a pipeline for intelligent text annotation of medical reports via artificial intelligence based Natural Language Processing workflows are provided. One or more embodiments described herein can regard a computer-implemented system comprising a memory that can store computer-executable components. The computer-implemented system can comprise a processor, operatively coupled to the memory, that executes the computer-executable components stored in the memory. The computer-executable components can comprise at least an artificial intelligence (AI) component that assigns relevant medical categories, comprising at least type and context categories, to cataloged data from one or more medical reports, by incorporating a Semi Automated Curation (SAC) workflow; an annotation component that generates AI based annotations for a cohort of medical reports of the one or more medical reports by incorporating an AI based Natural Language Processing (NLP) workflow; and a conversion component that converts, at least the AI based annotations, to standardized data formats.

### TECHNICAL FIELD

This application relates to a pipeline for intelligent text annotation of medical reports via artificial intelligence based Natural Language Processing workflows.

### BACKGROUND

Natural Language Processing (NLP) is used widely across various industries to assist computers and computing systems to understand written text and subsequently generate suggestions. However, this field is at a nascent stage in the healthcare industry. Healthcare companies have gradually aggregated a huge corpus of medical reports including prescriptions, lab reports, radiology reports, and patient information, and are facing challenges with corpus management, cataloging of medical report data for efficient cohort creation, text annotation methods for both structured and un-structured medical reports, and training and deployment of workflow models that address the challenges. Thus, there is a need for an intelligent workflow design that generates artificial intelligence (AI) based text annotation for medical reports using AI based NLP workflows.

### SUMMARY

The following presents a summary to provide a basic understanding of one or more embodiments of the invention. This summary is not intended to identify key or critical elements or delineate any scope of the different embodiments or any scope of the claims. Its sole purpose is to present concepts in a simplified form as a prelude to the more detailed description that is presented later.

An embodiment includes a computer-implemented system, comprising: a memory; and a processor that executes computer-executable components stored in the memory. The computer-executable components comprise: an AI component that assigns relevant medical categories, comprising at least type and context categories, to cataloged data from one or more medical reports, by incorporating a Semi Automated Curation (SAC) workflow, an annotation component that generates AI based annotations for a cohort of medical reports of the one or more medical reports by incorporating an AI based NLP workflow, a conversion component that converts, at least the AI based annotations, annotations generated by human entities, or a combination thereof, to standardized data formats, and a machine learning component that uses completed annotations to train the AI based NLP workflow via neural network models, to enhance accuracy of the AI based NLP workflow.

Another embodiment includes a computer-implemented method. The computer-implemented method comprises: assigning, by a system operatively coupled to a processor, relevant medical categories, comprising at least type and context categories, to cataloged data from one or more medical reports, by incorporating an SAC workflow, generating, by the system, AI based annotations for a cohort of medical reports of the one or more medical reports by incorporating an NLP based annotation workflow, converting, by the system, at least the AI based annotations, annotations generated by human entities, or a combination thereof, to standardized data formats, and training, by the system, the AI based NLP workflow via neural network models, using completed annotations, to enhance accuracy of the AI based NLP workflow.

Another embodiment includes a computer program product. The computer program product comprises a computer readable storage medium having program instructions embodied therewith. The program instructions are executable by a processor to cause the processor to: assign, by the processor, relevant medical categories, comprising at least type and context categories, to cataloged data from one or more medical reports, by incorporating an SAC workflow, generate, by the processor, AI based annotations for a cohort of medical reports of the one or more medical reports by incorporating an NLP based annotation workflow, convert, by the processor, at least the AI based annotations, annotations generated by human entities, or a combination thereof, to standardized data formats, and training, by the processor, the AI based NLP workflow via neural network models, using completed annotations, to enhance accuracy of the AI based NLP workflow.

### DESCRIPTION OF DRAWINGS

FIG. 1 illustrates a block diagram of an example, non-limiting computer-implemented system that facilitates integration of AI based NLP workflows for intelligent text annotation of medical reports in accordance with one or more embodiments described herein.

FIG. 2 illustrates a process diagram of an example, non-limiting computer-implemented system that facilitates integration of AI based NLP workflows for intelligent text annotation of medical reports in accordance with one or more embodiments described herein.

FIG. 3 illustrates a flow chart representation for exporting annotations to a standardized Mcode format in accordance with one or more embodiments described herein.

FIG. 4 illustrates an example of an FHIR bundle format in accordance with one or more embodiments described herein.

FIG. 5 illustrates an annotation dashboard that displays a completion status for annotations in accordance with one or more embodiments described herein.

FIG. 6 illustrates an annotation dashboard in accordance with one or more embodiments described herein.

FIG. 7A illustrates a data augmentation process that facilitates completion of annotations for medical reports in accordance with one or more embodiments described herein.

FIG. 7B illustrates a detailed view of the number of occurrences of entities in a pool of annotated data in accordance with one or more embodiments described herein.

FIG. 8 illustrates a sequence diagram for exporting and utilizing annotations of medical reports for training AI based NLP workflows in accordance with one or more embodiments described herein.

FIG. 9 illustrates a flow diagram of an example, non-limiting computer-implemented method that facilitates integration of AI based NLP workflows for intelligent text annotation of medical reports in accordance with one or more embodiments described herein.

FIG. 10 illustrates a block diagram of an example, non-limiting computer-implemented method that facilitates integration of AI based NLP workflows for intelligent text annotation of medical reports in accordance with one or more embodiments described herein.

FIG. 11 is a schematic block diagram illustrating a suitable operating environment; and

FIG. 12 is a schematic block diagram of a sample-computing environment.

### DETAILED DESCRIPTION

The following detailed description is merely illustrative and is not intended to limit embodiments and/or application or uses of embodiments. Furthermore, there is no intention to be bound by any expressed or implied information presented in the preceding Background section, Summary section or in the Detailed Description section.

The disclosed subject matter is directed to systems, computer-implemented methods, and/or computer program products that provide a pipeline for integrating AI based NLP workflows for intelligent text annotation of medical reports. In various embodiments, the disclosed techniques can be integrated for all healthcare modalities for which medical reports and/or machine logs are generated, including, but not limited to, X-Rays, Computerized Tomography scans (CT scans), Magnetic Resonance Imaging (MRI), Positron Emission Tomography-Computed Tomography (PET/CT), PET/MRI, mammography, and ultrasound. In other embodiments, the disclosed techniques can also be extended to non-medical domains, including machine log files, fault analyses.

The primary challenge addressed by the invention is that it can provide a pipeline for generating annotations for medical reports wherein the pipeline can integrate AI based NLP workflows to generate AI based annotations, and wherein the pipeline can generate annotations through human effort. The pipeline can further integrate additional AI based workflows to assist human entities to enhance accuracy of the AI based NLP workflows, and to continuously improve quality of subsequent AI based annotations. In various embodiments, the pipeline can assist the human entities to generate new AI based NLP workflows for new use cases, wherein the AI based data cataloging and text annotation of medical reports towards AI based NLP workflows form the unique selling proposition of the invention.

In various embodiments discussed herein, medical reports belonging to one or more patients can be stored in a data lake such that data from the medical reports can be cataloged using AI based data cataloging workflows. Subsequently, SAC workflows can be applied to the cataloged medical reports to generate additional insights and intelligence on the medical reports, including extracting sentiments and assigning relevant medical categories comprising type and context categories, to make the medical reports searchable. Medical reports can be filtered, based on assignment of the relevant medical categories, by subdomains comprising radiology, cardiology, pulmonology, surgery, or other medical subdomains, relevant to a use case, to generate a cohort of medical reports belonging to the one or more patients for the use case. Based on the cohort generation, the pipeline can facilitate intelligent selection of AI based NLP workflows that can generate AI based annotations for medical reports in the cohort of medical reports. The AI based annotations can be evaluated by human annotators and human curators to ensure that the medical reports in the cohort of medical reports are fully annotated and the completed annotation can be used to train the AI based NLP workflows for accuracy in subsequent applications.

The various embodiments discussed herein can facilitate extraction of maximum possible annotation information by human entities, by providing interactive displays that provide annotation and project completion metrics at each stage of a project, to aid human entities associated with the project, to fully annotate one or more medical reports. Hovering a cursor over displayed text on the interactive displays can further display hints and additional information about the displayed text that can be used by the human entities to identify missing annotations in addition to manual annotation of the displayed text. The various embodiments discussed herein can further facilitate exporting AI based annotations as well as manual annotations generated to standardized data formats for integrating annotated data in a medical system.

In the various embodiments discussed herein, the term “pipeline” can imply a workflow wherein multiple AI based workflows are stacked in a format such that the outcomes of one or more AI based workflows can be ingested by multiple other AI based workflows to achieve an end goal. In the various embodiments discussed herein, the pipeline can integrate multiple AI based workflows comprising at least AI based data cataloging workflows, SAC workflows, AI based NLP workflows, AI based completion workflows, and other AI based workflows wherein each workflows performs a specific task.

The technical advantages of the invention can include integration of AI solutions at each stage of data ingestion and annotation, intelligent annotation management, generation of custom AI based NLP workflows that can cater to different end uses, and continuous evaluation of AI based NLP workflows based on feedback by human entities on AI based annotations. The pipeline can abstract the model level annotation process during generation of AI based annotations and data ingestion, provide text-based annotation at scale, and allow project managers, annotators, and curators to visualize the progress of annotations and the various class groups involved in the annotated text. The invention can also be integrated on multi-tenant scalable platforms such as such as Edison AI workbench.

The commercial advantages of the invention can include efficient data cataloging for big data such as a large corpus of medical reports, and extension of the platform to non-medical data, such as machine data, with efficient modification of workflows. Further, the invention can be efficient in terms of time, cost, and manpower, wherein the invention can provide speedy text annotations and by reducing dependencies on human annotators for regular and specialized medical reports. The invention can efficiently generate AI based NLP workflows that can be readily available for use by end systems such as electronic medical records (EMRs), and the invention can offer a pool of rich variety of annotated data.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

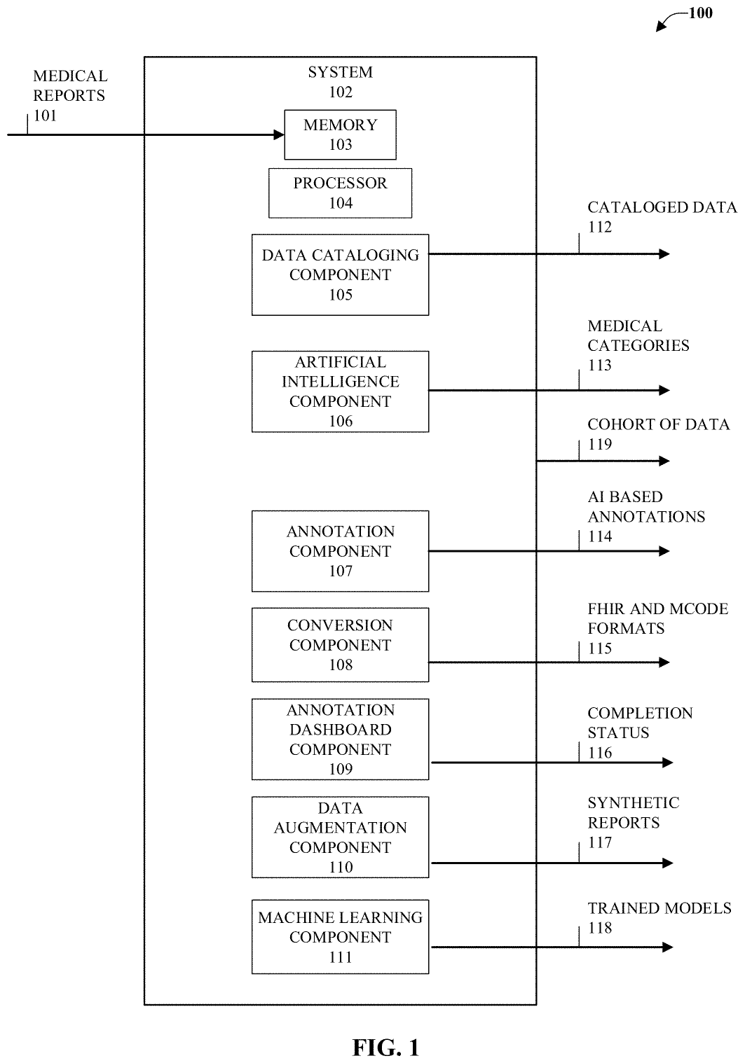

FIG. 1 illustrates a block diagram of an example, non-limiting computer-implemented system that facilitates integration of AI based NLP workflows for intelligent text annotation of medical reports in accordance with one or more embodiments described herein. FIG. 1 can comprise system 102 that can further comprise memory 103, processor 104, data cataloging component 105, artificial intelligence component 106, annotation component 107, conversion component 108, annotation dashboard component 109, data augmentation component 110, and machine learning component 111. It is to be appreciated that “artificial intelligence component” and “AI component” can be used interchangeably for one or more embodiments discussed in this application. In some embodiments, environment 100 can be a hospital whereas in other embodiments environment 100 can be a healthcare enterprise, private clinic, or another medical facility. Environment 100 can comprise system 102 wherein system 102 can be a computer-implemented system that can assist environment 100 to generate AI based annotations for data contained in the medical reports, wherein the AI based annotations can be used to create new AI based NLP workflows based on use cases and to train existing AI based NLP workflows to improve their accuracy.

Memory 103 can store medical reports 101 in a data lake. In some embodiments, medical reports 101 can belong to one patient, whereas in other embodiments, medical reports can belong to multiple patients. In an embodiment, medical reports 101 can be generated at a medical testing laboratory, hospital, and/or clinic located internal to environment 100, whereas in other embodiments, the medical report can be generated at a medical testing laboratory, hospital, and/or clinic located external to environment 100. Medical reports 101 can be generated as a result of prior workflows that can include radiology workflows, oncology workflows, and/or other healthcare related workflows. For example, a patient can undergo an oncology workflow that can produce medical reports comprising prescription medication reports, notes from a medical practitioner, surgery recommendations, follow up visit recommendations, and chemotherapy recommendations. Medical reports 101 can be one or more EMRs that can be uploaded in a variety of file formats including text files (TXT), JavaScript Object Notation (JSON), Comma-separated values (CSV), Portable Document Format (PDF), HyperText Markup Language (HTML), and Compact Disk Audio (CDA).

Data cataloging component 105 can catalog medical reports 101, by incorporating an AI based data cataloging workflow, upon ingestion of medical reports by system 102 such that metadata can be extracted from medical reports 101 to generate cataloged data 112. Artificial intelligence component 106 can select and apply SAC workflows to cataloged data 112 to assign medical categories 113 relevant to cataloged data 112 of medical reports 101 to segregate and curate cataloged data 112. Medical categories 113 can comprise type and context categories comprising radiological, oncological, cardiological, pulmonological, biomarker-based categories, and/or other relevant medical categories. Medical categories 113 can further comprise standard codes comprising Systemized Nomenclature of Medicine-Clinical Terms (SNOMED-CT), International Classification of Diseases-Tenth revision (ICD-10), RxNorm, Logical Observation Identifiers Names and Codes (Loinc), and Current Procedural Terminology (CPT). Medical categories 113 can make medical reports 101 searchable within the data lake of memory 103 such that a data scientist or a project manager can search through the data lake to create a cohort of data 119 comprising medical reports belonging to a specific subdomain or other medical category of medical categories 113. For example, a project manager can choose to create a cohort of cardiology based medical reports with the end goal of generating a new AI based NLP workflow for cardiology use cases. To generate the cohort of medical reports, the project manager can select the cardiology subdomain of medical categories 113 on a computer device and system 102 can produce a list of medical reports belonging to the cardiology subdomain. The project manager can select specific reports from the list of medical reports to generate the cohort of cardiology based medical reports for the use case.

Annotation component 107 can generate AI based annotations 114 for medical reports contained in the cohort of data 119. Artificial intelligence component 106 can identify an AI based NLP workflow to generate AI based annotations 114, based on a subdomain or other medical category of the cohort of data 119. For example, in reference to the preceding example wherein a project manager can create a cohort of cardiology based medical reports, artificial intelligence component 106 can auto select an AI based NLP workflow for cardiology, from one or more existing AI based NLP workflows comprising, surgery-based workflows, oncology-based workflows, and other workflows. In various embodiments, cohort of data 119 can belong to multiple subdomains and the artificial intelligence component 106 can select all relevant AI based NLP workflows for the subdomains. For example, a patient experiencing heart issues can undergo a cardiology investigation and a cardiovascular radiology investigation followed by surgery, and each investigation can generate one or more medical reports. A project manager can choose to create a cohort of medical reports for all the investigations of the patient, based on which, the artificial intelligence component 106 can select an AI based NLP workflow for cardiology, an AI based NLP workflow for cardiovascular radiology, and an NLP based workflow for surgery, to extract all potential AI based annotations for the subdomains in the cohort of medical reports.

Artificial intelligence component can incorporate an AI based similarity workflow to determine a degree of similarity between at least two or more medical reports of the cohort of data 119 such that annotations generated for a medical report can be copied to one or more other medical reports based on the degree of similarity. For example, cohort of data 119 can comprise medical reports that belong to only a pulmonology subdomain as well as medical reports that belong to a pulmonology and a surgery subdomain. Thus, annotations generated for the medical reports belonging to only the pulmonology subdomain can be copied to medical reports belonging to the pulmonology and surgery subdomains based on a degree of similarity between the two reports to determine relevant annotations to copy. Artificial intelligence component 106 can further provide a relationship extraction functionality such that the medical reports belonging to different subdomains (e.g., cardiology, radiology, etc.) in cohort of data 119 can be correlated and summarized to extract critical findings and determine a relationship between the medical reports.

AI based annotations 114 can be analyzed by one or more human annotators who can accept, reject, rectify, or otherwise modify AI based annotations 114, and wherein the human annotators can add manual annotations to AI based annotations 114 to extract all potential annotations for medical reports contained in cohort of data 119 and generate completed annotations. Herein, annotations can be assigned a completion status when all significant entities in the text of the medical reports can be considered annotated, and all relevant labels can be assigned to all of the significant entities. The manual annotations can comprise named entity recognition (NER), Part-of-speech (POS), relationships, and other NLP based annotations. The completed annotations can be further reviewed by a human curator with deeper domain understanding, wherein the human curator can select a set of completed annotations, from multiple sets of completed annotations generated by the one or more annotators, and wherein the human curator can mark the set of completed annotations as a final set of annotations that can be used for subsequent tasks required for the respective use case. The final set of annotations can also be used for ground truth generation to eventually assist a data scientist to train existing AI based NLP workflows for enhanced accuracy.

Annotations contained in the final set of annotations can be converted to standardized formats by conversion component 108, wherein the standardized formats can comprise Fast Healthcare Interoperability Resources and Minimal common oncology data elements formats 115 (FHIR and Mcode formats 115). The standardized formats can further comprise Computational Natural Language Learning (CoNLL) format, and the standardized formats can assist with integration of annotated data into a medical system such as environment 100. FHIR and Mcode format 115 can offer a patient view based on the completed annotations, wherein the patient view can display one or more medical conditions, recommended medications and other information pertaining to a patient's medical background as an interactive map of the patient's medical situation. Annotation dashboard component 109 can display the patient view on an annotation dashboard and artificial intelligence component 106 can provide a report linking functionality such that upon selecting an entity of the patient view on the annotation dashboard, a user of the annotation dashboard is redirected to a medical report of cohort of data 119 that the entity can be extracted from.

Annotation dashboard component 109 can further display, on the annotation dashboard, completion status 116 for annotations contained in medical reports of cohort of data 119 wherein completion status 116 corresponds to an amount of annotations extracted by human entities out of a total number of potential annotations detected by annotation component 107, for a respective medical category. Completion status 116 can be displayed as a percentage or as a number of completed annotations. Completion status 116 can provide to entities, information comprising the various categories of annotations detected by annotation component 107 in medical reports contained in cohort of data 119. Completion status 116 can further provide to the human entities, information regarding an amount of pending annotations for the various categories of annotations as well as missing annotations for certain categories, wherein the information can further assist the one or more human annotators to fully annotate medical reports contained in the cohort of data 119. Artificial intelligence component 106 can generate completion status 116 by incorporating an AI based completion workflow.

Data augmentation component 110 can generate synthetic reports 117, wherein synthetic reports 117 can comprise additional annotated versions of medical reports contained in cohort of data 119 that can provide additional AI based annotations for the human entities to analyze such that the medical reports can be fully annotated. Synthetic reports 117 can be generated by the human entities by clicking on a data augmentation tab on the annotation dashboard. Data augmentation component 110 can be assisted by the artificial intelligence component 106 to generate synthetic reports 117, and the data augmentation functionality can be user driven.

Completed annotations generated by human annotators and human curators can be used to generate a pool of annotations for ground truth generation to verify quality and accuracy of AI based annotations 114 generated by AI based NLP workflows. The pool of annotations can be exported to a machine learning pipeline wherein machine learning component 111 can use the pool of annotations to assist one or more data scientists to train existing AI based NLP workflows via a neural network model and/or to create new AI based NLP workflows for new use cases. The trained models 118 can be deployed as workflows that can be used to annotate new medical reports in a hospital setting such as environment 100.

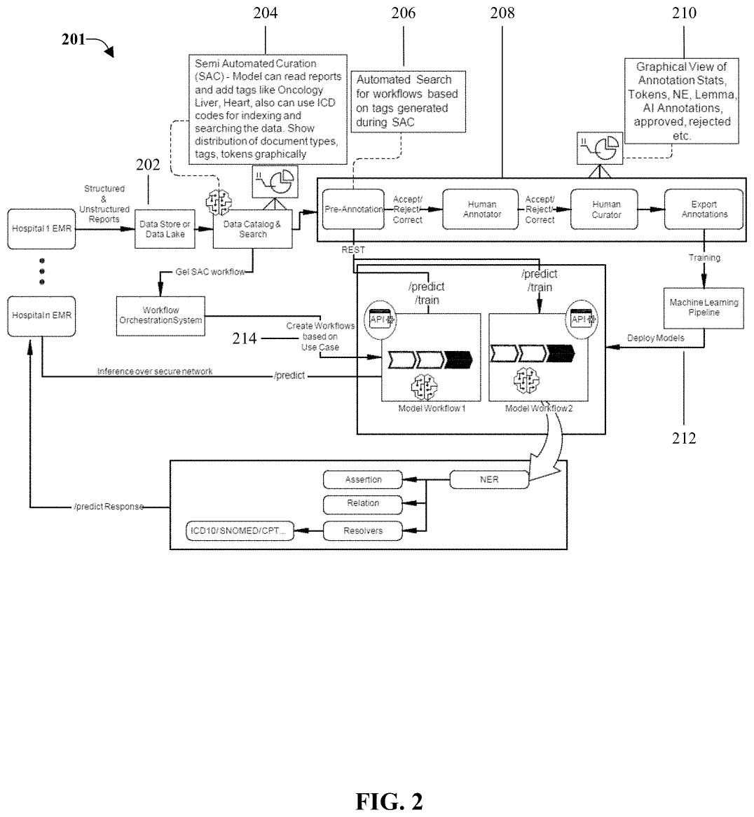

FIG. 2 illustrates a process diagram of an example, non-limiting computer-implemented system that facilitates integration of AI based NLP workflows for intelligent text annotation of medical reports in accordance with one or more embodiments described herein. FIG. 2 can comprise a pipeline 201 that can facilitate the generation of new AI based NLP workflows and training of existing AI based NLP workflows by employing system 102 of FIG. 1. Thus, FIG. 2 can be understood in conjunction with system 102 of FIG. 1. Repetitive description of like elements employed in respective embodiments is omitted for sake of brevity.

Pipeline 201 can facilitate integration of AI based workflows at each stage during ingestion of data to generate and attach tags comprising medical categories to medical reports to make the medical reports searchable within a database of a medical environment, provide AI based selection of AI based NLP workflows based on the generated tags, and use the AI based NLP workflows to generate AI based annotations for the medical reports. At 202, pipeline 201 can facilitate storage of medical reports 101 belonging to a patient in a data lake in memory 103 of system 102. At 204, the stored medical reports can be cataloged by data cataloging component 105. At 204, artificial intelligence component 106 can generate tags comprising medical categories 113 for cataloged data 112 of medical reports 101, using SAC workflows that can scan the reports to detect the type and context of the report and segregate data contained in the medical reports. Further at 204, the SAC workflows can be incorporated into the pipeline to create new AI based NLP workflows for new use cases. The data cataloging and generation of medical categories for medical reports 101 can make medical reports 101 more searchable in the database of system 102, and a data scientist can generate a cohort of data 119 comprising medical reports belonging to one or more specific medical categories. At 206, pipeline 201 can facilitate intelligent selection of the suitable AI based NLP workflow by artificial intelligence component 106, based on the specific medical categories contained in the cohort of data 119, for generation of AI based annotations 114 for the medical reports by annotation component 107. Step 206 can also be referred to as pre-annotation of data which can imply generation of AI based annotations 114 prior to evaluation of the AI based annotations and/or addition of manual annotations by human entities.

At 208, pipeline 201 can comprise an annotation workbench wherein AI based annotations 114 can be generated and evaluated by one or more human annotators who can accept, reject, correct and/or add additional annotations to the AI based annotations. The one or more human annotators can annotate entities, relations, assertions, co-reference resolutions, and/or temporals to respectively generate the ground truth for entity recognition, relationship extraction between pairs of entities, assertion status evaluation, and/or co-reference resolution analysis aimed towards training the AI based NLP workflows. The one or more human annotators can generate multiple sets of completed annotations that can be further evaluated by at least one human curator who can select a set of annotations to review, check for bias, and/or update the annotations to generate a final set of annotations that can be used for training AI based NLP workflows, based on the respective use case. At 204, pipeline 201 can also facilitate data augmentation by data augmentation component 110 that can generate synthetic reports 117 comprising additional annotated versions of medical reports contained in cohort of data 119, wherein synthetic reports 117 can provide additional data for the human entities to annotate and achieve finality of annotations within pipeline 201. The completed annotations can be used to train existing AI based NLP workflows at run time such that they can be continuously improved. The various AI models incorporated by pipeline 201 can thus determine a best arrangement of information contained in cohort of data 119 to generate a summary and a readable format of the information such that human entities can easily read the information and enhance best practices for training and generating AI based NLP workflows.

Annotations generated at each stage of the workbench at 208 can be displayed on an annotation dashboard, and the annotation dashboard can further display cataloged data 112, medical categories 113, a graphical view of the annotation statistics such as completion status 116, AI based annotations 114, as well as manual annotations added by the one or more human annotators. The annotations generated at 208 by the human entities and by the annotation component 107 can be converted to FHIR and Mcode formats 115 and to CoNLL format, by conversion component 108, upon completion of the annotation process. The FHIR format can correspond to generic reports whereas the Mcode format can correspond to oncology-based reports. In various embodiments, the set of completed annotations selected by a human curator can be further assessed by a project manager for the respective use case to generate a pool of annotations for ground truth generation. The set of completed annotations can be exported to machine learning pipeline 212 wherein machine learning component 111 can use annotations from the pool of ground truth annotations to assist a data scientist to train AI based NLP workflows via a neural network model. The data scientist can also use the annotations from the pool of ground truth annotations to create new AI based NLP workflows for new use cases, wherein the new AI based NLP workflows can be deployed to environments such as environment 100 for EMR use cases.

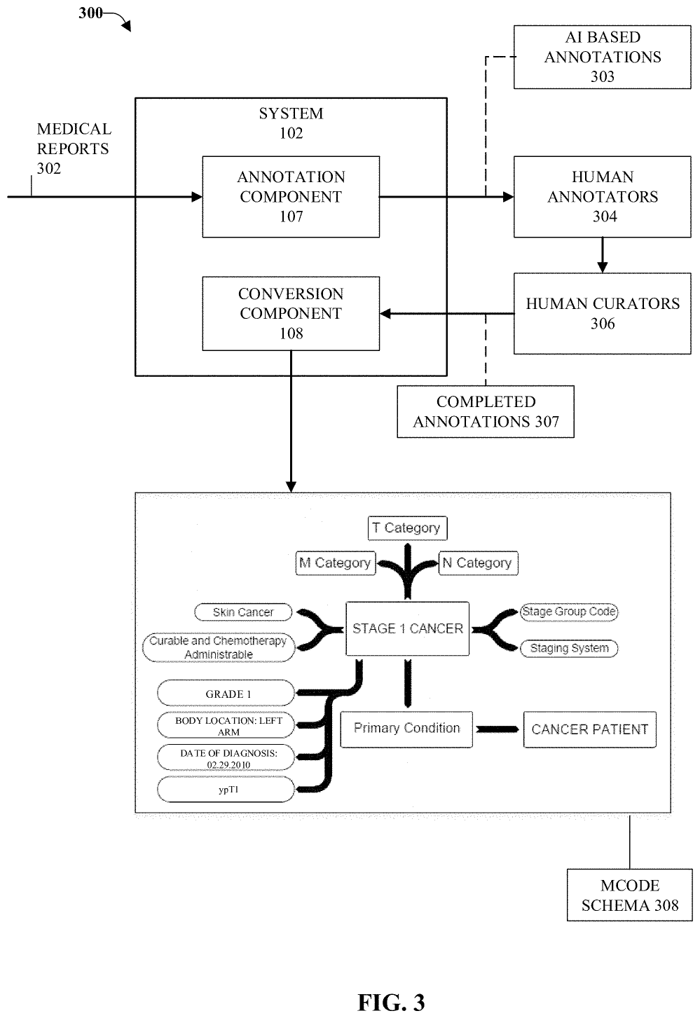

FIG. 3 illustrates a flow chart representation for exporting annotations to a standardized Mcode format in accordance with one or more embodiments described herein. FIG. 3 comprises system 102, annotation component 107, and conversion component 108 of FIG. 1. FIG. 3 further comprises environment 300, medical reports 302, AI based annotations 303, human annotators 304, human curator 306, and Mcode schema 308.

In some embodiments, environment 300 can be a hospital whereas in other embodiments, environment 300 can be a healthcare enterprise, private clinic, or another medical facility. Medical reports 302 can be received by environment 300 wherein the medical reports 302 can be stored, cataloged, and made searchable through assignment of relevant medical categories (e.g., subdomains such as surgery, standard codes, etc.), via AI based workflows, in accordance with one or more embodiments discussed herein. A data scientist can search through the database of system 102 to create a cohort of medical reports for the oncology subdomain based on the assignment of the relevant medical categories. The cohort of medical reports can be annotated by annotation component 107, wherein the annotation component 107 can generate AI based annotations 303 using an AI based NLP workflow for oncology, and wherein the AI based annotations 303 can be further evaluated by the human annotators 304 and subsequently by the human curators 306, to generate completed annotations 307 that can be exported to a machine learning pipeline for training new and existing AI based NLP workflows, for oncology. Herein, completed annotations 307 can imply a final set of annotations approved by one or more human curators represented by human curators 306, for further use for the respective use case. Thus, completed annotations can comprise annotations generated by the annotation component 107 as well as annotations generated by human entities.

Completed annotations 307 can be converted to an Mcode format by conversion component 108, wherein the Mcode format can be a standardized data format for oncological annotations to be integrated into a medical system such as environment 300. An Mcode format can represent a clinical entity as the stem of a tree and corresponding medical subcategories as branches of the tree, such as can be represented by Mcode schema 308. Mcode schema 308 can display a TNM staging mechanism for cancer, wherein the T category can correspond to a primary tumor, the N category can correspond to nodal involvement and M category can correspond to metastatic disease. The TNM staging mechanism can display a main category of cancer and related subcategories including, but not limited to, a grade of cancer, required treatments for the type of cancer, location of the cancer on the body, date of diagnosis, and pertinent oncological codes for the respective stage of cancer, wherein each subcategory can be a result of an independent medical investigation that can product multiple medical reports. It is to be appreciated that the subcategories depicted in Mcode schema 308 are for purely exemplary purposed and additional subcategories can be represented by an Mcode format. Thus, Mcode schema 308 can assist data scientists to view the medical categories and medical subcategories related to a patient for a respective use case as a connected map of all investigations and investigative results related to a medical condition of a patient.

In various embodiments, Mcode schema can be generated prior to generation of AI based annotations by annotation component 107. Herein, data scientists can use Mcode schema to verify accuracy of outputs generated by AI based NLP workflows, as a result of training by neural network models, by mapping outputs from the neural network models to the Mcode based bundle. Likewise, FHIR bundles can also assist data scientists to ensure that AI based NLP workflows created by them can be expected to generate AI based annotations with accuracy levels above a defined threshold. Mcode and FHIR formats can further assist the data scientists to enhance the quality of training new and existing AI based NLP models.

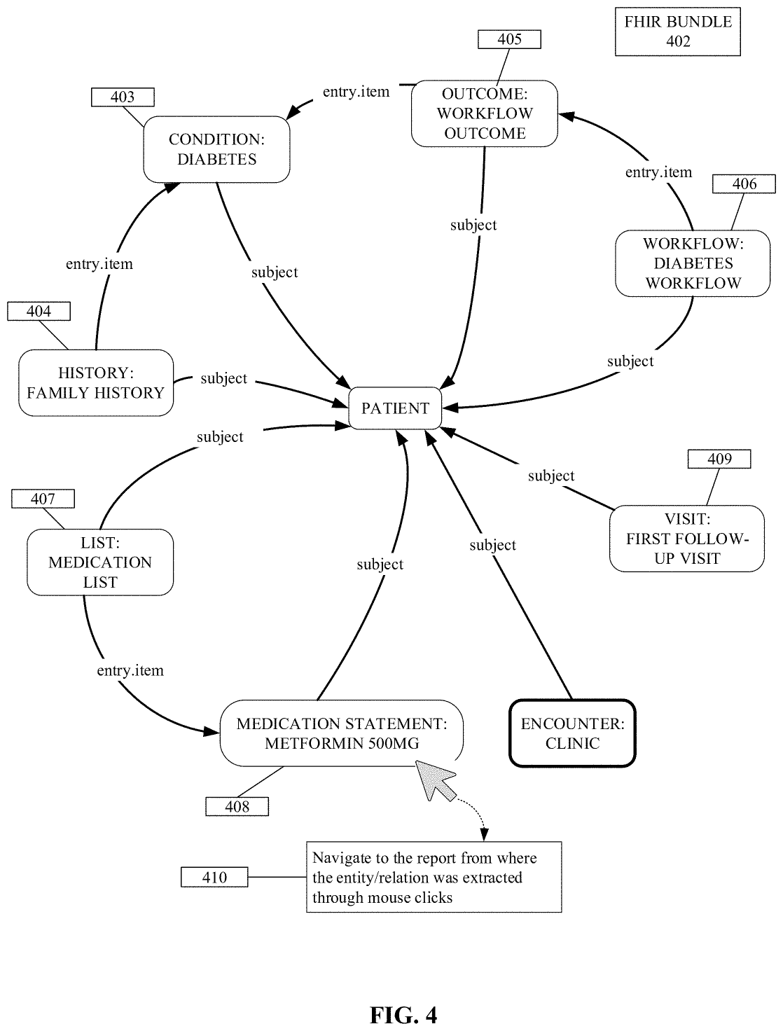

FIG. 4 illustrates an example of an FHIR bundle format in accordance with one or more embodiments described herein. Repetitive description of like elements employed in respective embodiments is omitted for sake of brevity.

In various embodiments discussed herein, annotations generated at an annotation workbench (e.g., annotation workbench 208 of FIG. 2) for medical reports of a patient can be converted to FHIR bundle 402 that can demonstrate a patient view of the exported annotations. The patient view can act as a knowledge map of the various medical conditions, medications, and other health related information about the patient in a user-friendly format as opposed to a raw data format such that human entities can infer critical medical information regarding the patient from FHIR bundle 402 without needing to read through medical reports pertaining to the patient. Thus, FHIR bundle 402 can provide a convenient way for the human entities to view a patient's ontology, and, as discussed in one or more embodiments herein, the FHIR bundle can determine relationships between critical results from various investigative workflows corresponding to the patient's medical condition. FHIR bundle 402 can be displayed on an annotation dashboard wherein it can be used as an interactive map by the human entities to extract additional information about the patient.

For example, FHIR bundle 402 can map a patient's diabetic condition at 403 to the patient's family history at 404. The diabetic condition can be further mapped as a workflow outcome at 405 of a diabetes workflow displayed at 406. FHIR bundle 402 can display a medication list at 407 and map it to the various medications recommended to the patient. For example, at 408, FHIR bundle 402 can map a medication statement to the medication list. Further at 409, information regarding a follow-up visit of the patient with a medical practitioner can also be mapped by FHIR bundle 402, The diabetic condition, workflow outcome, family history, medication list and the follow-up visit can be further mapped to the patient such that it can be possible to navigate to a medical report belonging to the patient through an entity displayed by FHIR bundle 402. For example, at 410, a user of the annotation dashboard can click on the medication statement at 408 via a cursor and the user can be redirected to the medical report comprising medication information for the patient. FHIR and Mcode formats can comprise a link to the Uniform Resource Identifier (URI) of the original medical report that can cause the original medical report to be downloaded and displayed in response to the user clicking on entities.

In various embodiments discussed herein, FHIR bundles such as FHIR bundle 402 can be generated prior to generation of AI based annotations by via an AI based NLP component, such that data scientists can use FHIR bundle to verify accuracy of outputs generated by AI based NLP workflows, as a result of training by neural network models, by mapping outputs from the neural network models to the FHIR bundles.

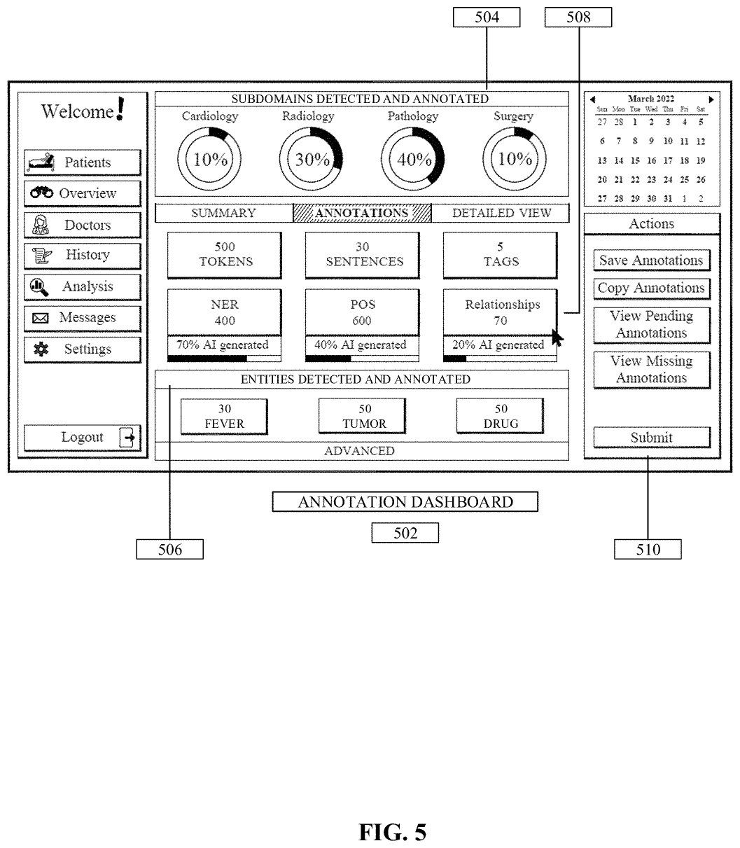

FIG. 5 illustrates an annotation dashboard that displays a completion status for annotations in accordance with one or more embodiments described herein. Repetitive description of like elements employed in respective embodiments is omitted for sake of brevity.

In an embodiment, a computer-implemented system can facilitate integration of AI based NLP workflows and additional AI based workflows for intelligent text annotation of medical reports, wherein AI based annotations can be generated for EMRs via AI based NLP workflows, and the AI based annotations can be evaluated and/or manipulated by human annotators and human curators to generate completed annotations. The AI based annotations and manual annotations generated by human entities, comprising human annotators and human curators, can be displayed on annotation dashboard 502, such that the human entities can use the displayed information and annotation statistics to further annotation tasks and extract maximum possible annotations from the medical reports.

Annotation dashboard 502 can display the medical subdomains detected by the computer-implemented system, in the medical reports, and a completion status of annotations for the individual medical subdomains, wherein the completion status can indicate to human entities, an amount of annotations generated by them for the individual medical subdomains out of a total amount of annotations detected by the computer-implemented system via AI based workflows. For example, at 504, annotation dashboard 502 can display a 10 percent (%) completion status for cardiology based annotations, a 30% completion status for radiology based annotations, a 40% completion status for pathology based annotations, and a 10% completion status for surgery based annotations. Annotation dashboard 502 can further display a completion status for NLP based annotations and for additional entities detected by the computer-implemented system via the AI based workflows. For example, at 506, annotation dashboard 502 can display that 30 annotations for the word “fever,” 50 annotations for the word “tumor,” and 50 annotations for the word “drug” have been generated by the human entities. At 508, annotation dashboard 502 can display that 400 NER annotations, 600 POS annotations, and 70 Relationships have been generated by the human entities.

Additionally, at 508, a percentage completion for the NLP based annotations can be displayed by annotation dashboard 502, wherein the percentage completion can indicate to the human entities that the number of entities generated by them can comprise a percentage of the total annotations detected for the particular medical category, by the AI based workflows. For example, annotation dashboard 502 can display a label such as: “70% AI generated,” for the 400 NER annotations that can be generated by human entities. This can imply that the 400 NER annotations comprise 70% of total NER annotations detected for the particular use case, and the percentage completion information can alert the human entities to the knowledge that additional annotations can be generated for the particular category of annotations. In this regard, annotation dashboard 502 can provide, at 510, additional tools for generation of completed annotations. At 510, the human entities can choose to view pending annotation, view missing annotations, and/or save the generated annotations to return to the annotation tasks at later time. Further at 510, the human entities can copy annotations for generated for a medical report of the medical reports to apply the copied annotations to another medical report based on a degree of similarity between the medical reports.

Thus, the completion status for annotations can assist the human annotators and curators to evaluate the progress of annotation tasks pertaining to medical reports for a respective use case. The completion status for annotations is generated via an AI based completion workflow by an AI component of the computer-implemented system, in accordance with one or more embodiments discussed herein. The AI component can further determine the degree of similarity between one or more medical reports via an AI based similarity workflow, in accordance with one or more embodiments described herein. An annotation dashboard component can facilitate the display of information pertaining to the annotations on an annotation dashboard such as annotation dashboard 502.

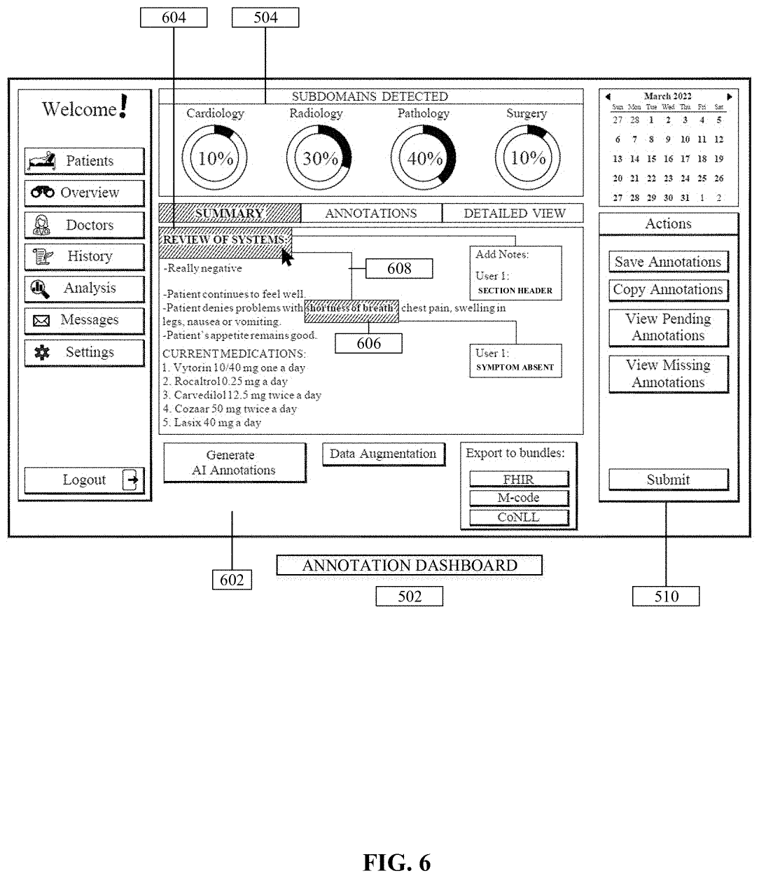

FIG. 6 illustrates an annotation dashboard in accordance with one or more embodiments described herein. Repetitive description of like elements employed in respective embodiments is omitted for sake of brevity.

FIG. 6 comprises annotation dashboard 502 of FIG. 5. As discussed in FIG. 5, annotation dashboard 502 can display percentage completion of annotations for various medical categories and subdomains and provide actionable steps to assist human annotators and human curators towards completion of annotation tasks for medical reports for a respective use case. Annotation dashboard can further display annotation distributions in terms of entities, assertions, relations, section headers, potential patient specific information, prescriptions of medications, signature of the concerned physician, information about the physician, and planning tools such as calendars.

Additionally, annotation dashboard 502 can provide a summary for a patient, and human entities can click on the summary tab to add additional annotations for entities in the summary. At 602 of annotation dashboard 502, the human annotators can generate additional AI based annotations for medical reports, wherein upon generation of the AI based annotations, annotation dashboard 502 can display, at 504, medical subdomains detected by a computer-implemented system, in the medical reports, and a completion status of annotations for the individual medical subdomains, in accordance with FIG. 5.

Annotation dashboard 502 can further allow human annotators to add additional annotations. For example, at 604, a human annotator can select the phrase “review of systems” and right click on the phrase to annotate it as “Section header.” Similarly, the phrase “shortness of breath” can be annotated by the human annotator as “Symptom absent.” At 608, the annotator can further provide a link between the two annotations wherein the link can be descriptive of the relationship between the two annotations. Similarly, the human annotator can add additional labels to annotate the patient specific information and subsequently check the percentage completion status of the respective annotations, wherein the percentage completion of an annotation can be updated by an annotation dashboard component of the computer-implemented system upon augmentation of the particular annotation. Thus, the human annotators can view the completion status of annotations for individual medical categories and entities and use the information to add additional annotations to complete the annotation tasks.

At 602, the human annotators can further generate AI based synthetic data by selecting a data augmentation option. While the data augmentation for medical reports can ideally be performed prior to the generation of AI based annotations, the human annotators have the option to use the data augmentation functionality upon generation of the AI based annotations if the human annotators can determine, based on the percentage completion status of the annotations, that certain categories of annotations can require additional annotations for completion. At 602, the human annotators can export completed annotations to FHIR and Mcode formats for visualization of annotated data via FHIR and Mcode bundle, respective, in accordance with one or more embodiments described herein. Additionally, at 602, the huma annotators can export completed annotations to a CoNLL format with entity information in Inside, outside, beginning (IOB/BIO) tagging or Parts of Speech (POS) tagging formats. The human annotators can add annotator notes for every annotation displayed on annotation dashboard 502, wherein the annotator notes can be exported, alongside the completed annotations, to CoNLL, FHIR, and Mcode formats.

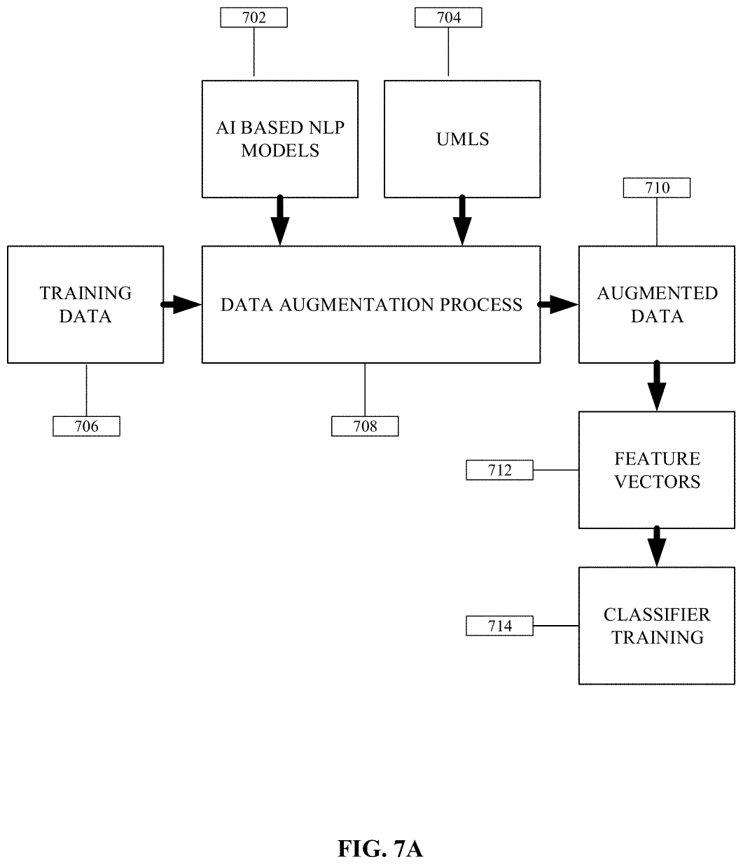

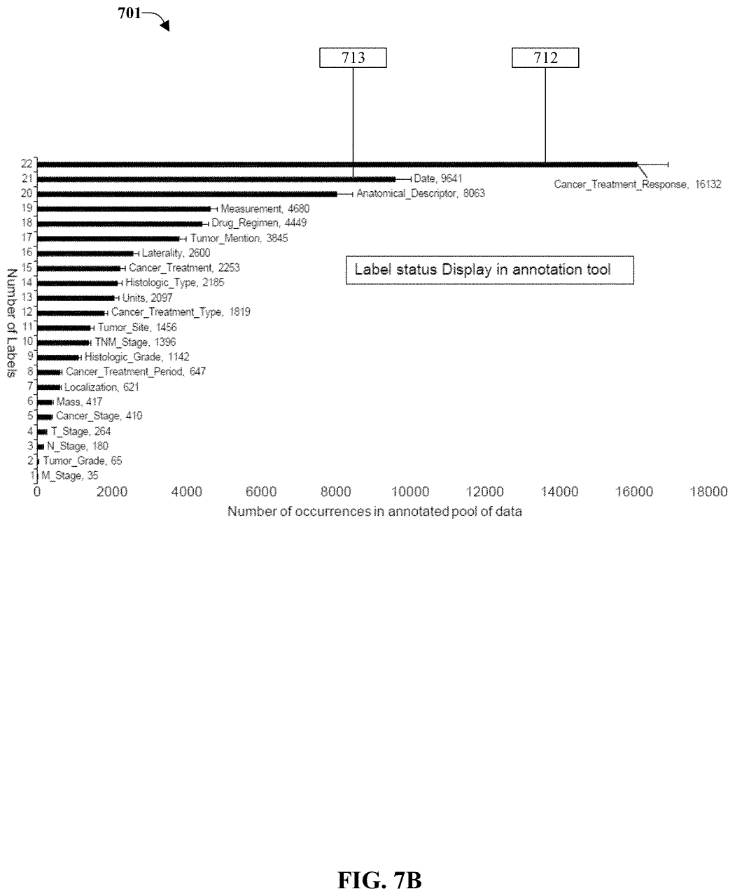

FIG. 7A illustrates a data augmentation process that facilitates completion of annotations for medical reports in accordance with one or more embodiments described herein. Repetitive description of like elements employed in respective embodiments is omitted for sake of brevity.

In various embodiments, medical reports contained in a cohort of data (e.g., cohort of data 119 of FIG. 1) can be annotated by an AI based NLP workflow to generate AI based annotations that can be evaluated and manipulated by human entities. In various embodiments, the medical reports can be subjected to a data augmentation process, such as data augmentation process 708, prior to generation of AI based annotations, to assist the human entities with the annotation process so that a maximum number of potential annotations can be extracted from the medical reports. Data augmentation process 708 can run as a background process to the annotation process (e.g., such as in annotation workbench at 208 of FIG. 2), and data augmentation process 708 can be initiated by the human entities via mouse clicks on a data augmentation option on an annotation dashboard (e.g., data augmentation tab of annotation dashboard 502 in FIG. 6). Upon initiation of data augmentation process 502, a data augmentation algorithm can be triggered in a computer-implemented system (e.g., system 102 of FIG. 1). Data augmentation process 708 can utilize different AI based NLP models represented as AI based NLP models 702, wherein the models can be trained on word embeddings, on sentence embeddings and on key words present in the text of training data 706, to generated augmented data 710. Data augmentation process 708 can incorporate Unified Medical Language System 704 (UMLS 704) for generating augmented data 710, wherein augmented data 710 can comprise additional sets of AI based synthetic data, based on the medical reports in the cohort of data.

For example, a sentence in a medical report can say: “The patient has right chest pain,” based on which, data augmentation process 708 can generate multiple synthetic sentences of a similar nature such as “The patient has left chest pain,” “The patient has chronic chest pain,” or “The patient was suffering from chronic chest pain for the past 4 days.” Thus, data augmentation process 708 can generate augmented data 710 comprising synonyms for words and sentences, wherein the augmented data 710 can be based on a syntactic approach, a synonym-based approach, or a combination of syntactic and synonym-based approach. Augmented data 710 can assist the human entities to expand the amount of useful information contained in a pool of ground truth annotations generated as an end product by an annotation workbench (e.g., annotation workbench at 208 of FIG. 2), for training AI based NLP workflows for enhancing the accuracy of the AI based NLP workflows. Augmented data 710 can further be used for generating feature vectors 712 and for classifier training 714.

FIG. 7B illustrates a detailed view of the number of occurrences of entities in a pool of annotated data in accordance with one or more embodiments described herein. Repetitive description of like elements employed in respective embodiments is omitted for sake of brevity.

As discussed in various embodiments herein, an annotation dashboard (e.g., annotation dashboard 502 of FIG. 5) can display a completion status of annotations generated for medical reports by an AI based NLP workflow at an annotation workbench (e.g., annotation workbench at 208 of FIG. 2). A human annotator evaluating the AI based annotations, can choose a type of annotation view for the evaluation. The human annotator can choose a subdomain based view (e.g., as illustrated by annotation dashboard 502 of FIG. 5) or the human annotator can choose a detailed view 701 as illustrated by FIG. 7B by selecting a “detailed view” tab (e.g., “detailed view” tab on annotation dashboard 502 of FIG. 5) on an annotation dashboard. Detailed view 701 can display a graph of the number of labels generated for all clinical entities detected in the medical reports by the AI based NLP workflow versus the number of occurrences of entities in the pool of annotated data. For example, at 712, the detailed display can indicate that annotations comprise 16,132 occurrences in the annotated data mention a cancer treatment response and, at 713, the detailed display can indicate that 9641 occurrences in the annotated data mention a date. Detailed view 701 can assist human annotators to identify and fix skewness in annotated data from medical reports, and detailed view 701 can further assist data scientists to identify efficient ways to train the AI based NLPs.

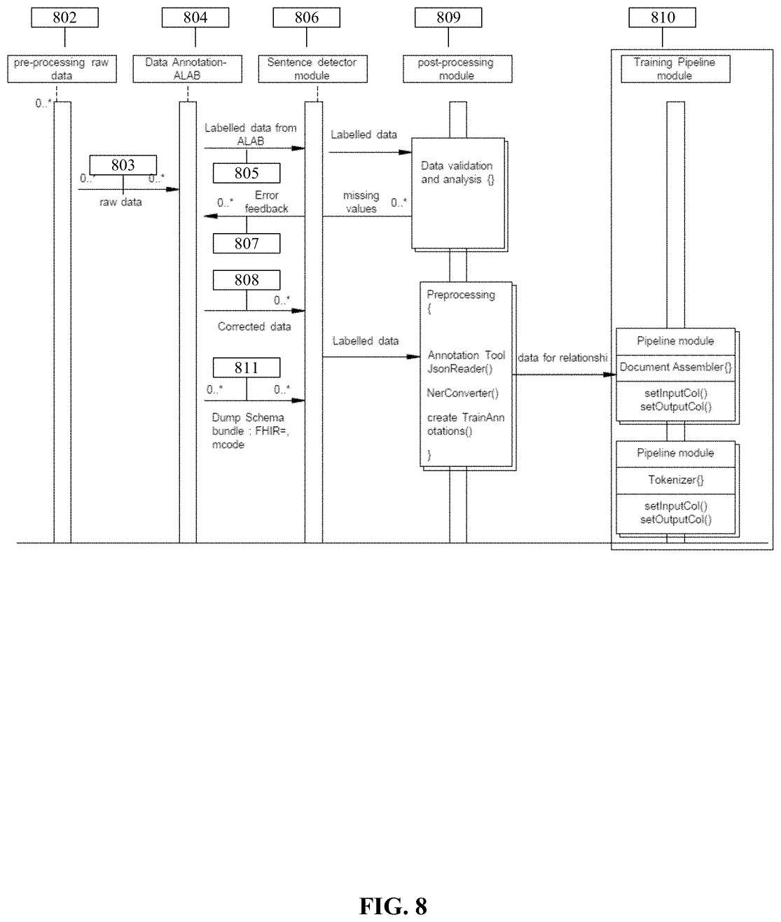

FIG. 8 illustrates a sequence diagram for exporting and utilizing annotations of medical reports for training AI based NLP workflows in accordance with one or more embodiments described herein.

In an embodiment, medical reports for a patient can be generated at 802, at a medical testing laboratory, hospital, and/or clinic, as a result of various clinical investigative workflows (e.g., radiology workflows, oncology workflows, etc.). The medical reports can comprise raw data including different diagnoses for the patient, scanning techniques used for the different diagnoses, and treatment plans outlined for the patient based on assessment of the diagnoses. At 803, the medical reports comprising the raw data can be uploaded to a computer-implemented system that facilitates integration of AI based NLP workflows for intelligent text annotation of medical reports. As discussed in one or more embodiments, the raw data can be uploaded to the computer-implemented system in multiple formats including TXT, CSV, and PDF. The medical reports can be stored, cataloged, and assigned relevant medical categories via the SAC workflows to make the medical reports searchable within a database of a medical environment (e.g., hospitals, clinics, medical testing laboratories). The searchable medical reports can be utilized by a data scientist to create a cohort of reports belonging to specific subdomains (e.g., oncology, pulmonology, surgery).

The cohort of medical reports can be annotated in a data annotation laboratory (ALAB) at 804, wherein AI based annotations and additional manual annotations by human entities can be generated for the medical reports to generate a set of completed annotations. The set of completed annotations, comprising the AI based annotations and/or the manual annotations, can be detected by a sentence module detector 806 as labelled data 805 from the ALAB. Sentence module detector 806 can determine the type of labels that can be present in labelled data 805 and determine a completion status for the annotations, wherein the completion status can correspond to an amount of annotations extracted by human entities out of a total number of potential annotations detected by an annotation component, for a respective medical category. Annotations detected as incomplete by the sentence module detector can be sent to the ALAB as error feedback 807. Error feedback 807 can be displayed on an annotation dashboard (e.g., annotation dashboard 502 of FIG. 5) as a list of annotations with percentage completions that can describe the medical categories of entities occurring in the medical reports and the amount of completely annotated, pending annotations and/or missing annotations for the medical categories. The human entities at the ALAB can modify the annotations based on error feedback 807 to correct and/or complete the annotations, wherein the completed annotations can be detected by the sentence module detector as corrected data 808.

Post-processing module 809 can store corrected data 808 in multiple formats, including JSON, CSV and a Microsoft Excel Open Extensible Markup Language Spreadsheet (XLSX) format, to construct a pool of ground truth annotations for training AI based NLP workflows by neural network models. The pool of ground truth data can include the AI based annotations and manual annotations by human entities. The manual annotations can include additional annotations that can appear on the annotation dashboard as popups in response to a user of the annotation dashboard hovering a cursor on medical text displayed on the annotation dashboard. Training pipeline module 810 can use the pool of ground truth to train the AI based NLP workflows. Corrected data 808 can also be exported to standardized formats, including CoNLL, FHIR and Mode formats. For exporting annotations to Mcode and FHIR formats, the annotations can be stored in Unstructured Information Management Architecture-Computer Algebra System (UIMA CAS), and multiple novel adapters can be used to convert the entities identified by the AI based NLP workflow into Mcode and FHIR formats.

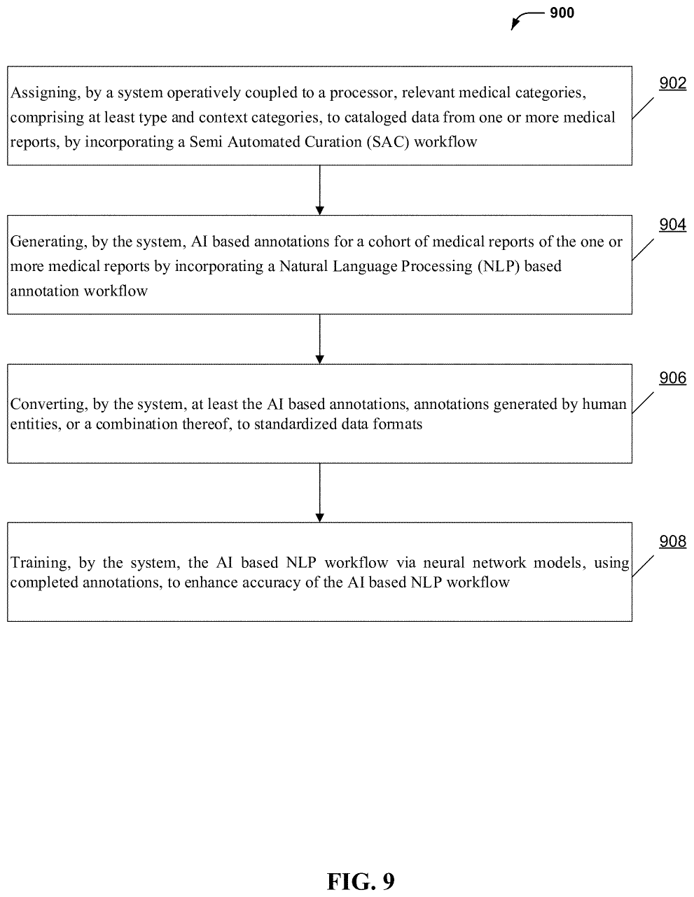

FIG. 9 illustrates a flow diagram of an example, non-limiting computer-implemented method that facilitates integration of AI based NLP workflows for intelligent text annotation of medical reports in accordance with one or more embodiments described herein.

In an embodiment, environment 900 can comprise a computer-implemented method. At 902, the computer-implemented method can facilitate assigning, by a system operatively coupled to a processor, relevant medical categories, comprising at least type and context categories, to cataloged data from one or more medical reports, by incorporating a Semi Automated Curation (SAC) workflow. At 904, the computer-implemented method can facilitate generating, by the system, AI based annotations for a cohort of medical reports of the one or more medical reports by incorporating a Natural Language Processing (NLP) based annotation workflow. At 906, the computer-implemented method can facilitate converting, by the system, at least the AI based annotations, annotations generated by human entities, or a combination thereof, to standardized data formats. At 908, the computer-implemented system can facilitate training, by the system, the AI based NLP workflow via neural network models, using completed annotations, to enhance accuracy of the AI based NLP workflow.

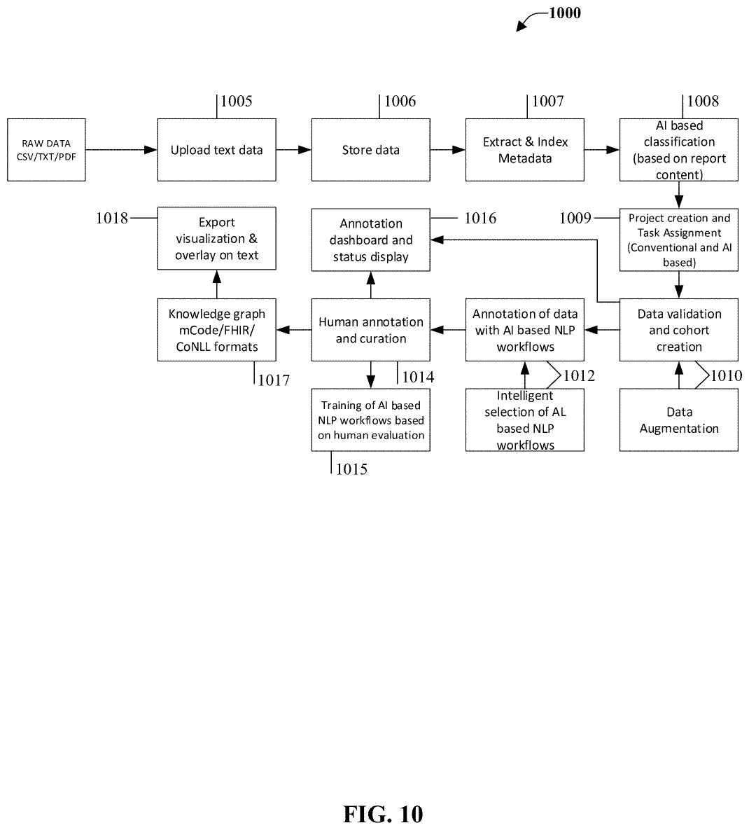

FIG. 10 illustrates a block diagram of an example, non-limiting computer-implemented method that facilitates integration of AI based NLP workflows for intelligent text annotation of medical reports in accordance with one or more embodiments described herein. Repetitive description of like elements employed in respective embodiments is omitted for sake of brevity. In some embodiments, environment 1000 can be a hospital whereas in other embodiments, environment 1000 can be a healthcare enterprise, private clinic, or another medical facility. Environment 1000 can comprise a computer-implemented method that can assist environment 1000 to generate AI based annotations for medical reports, wherein the AI based annotations can be used to train existing AI based NLP workflows to improve their accuracy, and to create new AI based NLP workflows for new use cases.

At 1005 of the computer-implemented method, medical reports of a patient can be uploaded to the system in a variety of file formats including TXT, JSON, CSV, PDF, HTML, and CDA. At 1006, the medical reports can be stored in the memory of a computer-implemented system operatively coupled to a processor. At 1007, the medical reports can be cataloged such that meta data can be extracted from the medical reports stored in the memory. At 1008 SAC workflows can be applied to the medical reports, based on the cataloging, to assign relevant medical categories to the cataloged medical reports. The medical categories can comprise radiological, oncological, cardiological, pulmonological, biomarker-based categories, and/or other relevant medical categories. The standard codes can comprise SNOMED-CT, ICD-10, RxNorm, Loinc, and CPT. The medical categories can make the medical reports searchable within a database of the computer-implemented system. At 1009, a project manager can create a new project based on a use case, wherein the use case can comprise creation of a new AI based NLP workflow or training of an existing NLP based workflow. For example, the project manager can create a project for generating a new AI based NLP workflow that can detect radiological reports, or the project manager can create a project for improving the accuracy of an existing AI based NLP workflow for a cardiology subdomain.

Subsequently, a data scientist can search through the database of the computer-implemented system to create a cohort of data comprising medical reports with specific medical categories, at the data validation and cohort creation stage 1010. At 1010, the method can further comprise data augmentation where additional annotated versions of the medical reports contained in the cohort of data can be generated. For example, a cohort of medical reports created by a data scientist can comprise 50 medical reports, and the data augmentation function can facilitate the creation of additional data sets of the 50 medical reports, comprising synthetic data based on the 50 medical reports, such that the synthetic data can be used by human entities to extract maximum possible categories of annotations and maximum possible annotations for the various categories to generate a rich pool of ground truth annotations for training AI based NLP workflows via machine learning, and for enhancing the quality of the training. At 1012, AI based annotations can be generated for the medical reports contained in the cohort of data generated by the data scientist. At 1012, the computer-implemented method can also facilitate selection of the suitable AI based NLP workflow, based on the medical categories assigned to the medical reports contained in the cohort of data, for generating the AI based annotations. At 1014, the AI based annotations can be subsequently analyzed by one or more human annotators who can accept, reject, correct and/or add additional annotations to the AI based annotations to generate a completed annotations. The annotations provided by the one or more human annotators can comprise NLP based annotations such as NER, POS, relationships, and/or other NLP based annotations. The annotations generated by the one or more human annotators can be further reviewed by one or more human curators, wherein the human curator can select a set of completed annotations, from multiple sets of completed annotations generated by the one or more annotators, and wherein the human curator can mark the set of completed annotations as a final set of annotations that can be used for subsequent tasks required for the respective use case.

At 1015, the AI based NLP workflow used for generating the AI based annotations can be trained for improved accuracy. For example, the AI based NLP workflow can incorrectly predict a certain section of text contained in one of the medical report as a “document header.” A human annotator can review the prediction and the human annotator can decide that the “document header” can be correctly predicted as the “document main text,” and based on the observation, the human annotator can choose to have the AI based NLP model trained for enhanced accuracy in generating AI based annotations for new use cases. Data generated for the medical reports, including, but not limited to, the medical categories, augmented versions of medical reports, AI based annotations, and a completion status of annotations, can be displayed on an annotation dashboard at 1016. The annotation dashboard can display the completion status of the annotations alongside the status of the project, which can assist a project manager associated with the respective use case to drive the respective project towards completion. At 1017, a set of completed annotations as approved by one or more human curators and/or the project manager can be exported to CSV based standardized formats including FHIR, Mcode and/or CoNLL. At 1018, the exported data can be visualized as FHIR bundles, Mcode schema, and/or other mapping, and the text can be overlayed for displaying relevant sections of the treatment guidelines with clinical markers such that the relevant sections of the treatment guidelines can be presented as boxes of text at an annotation dashboard, and at least one corresponding clinical marker can be overlayed with each of the boxes of text. In accordance with one or more embodiments discussed herein, a user of the annotation dashboard can click on an entity on the annotation dashboard, to view the respective medical report that the entity was extracted from.

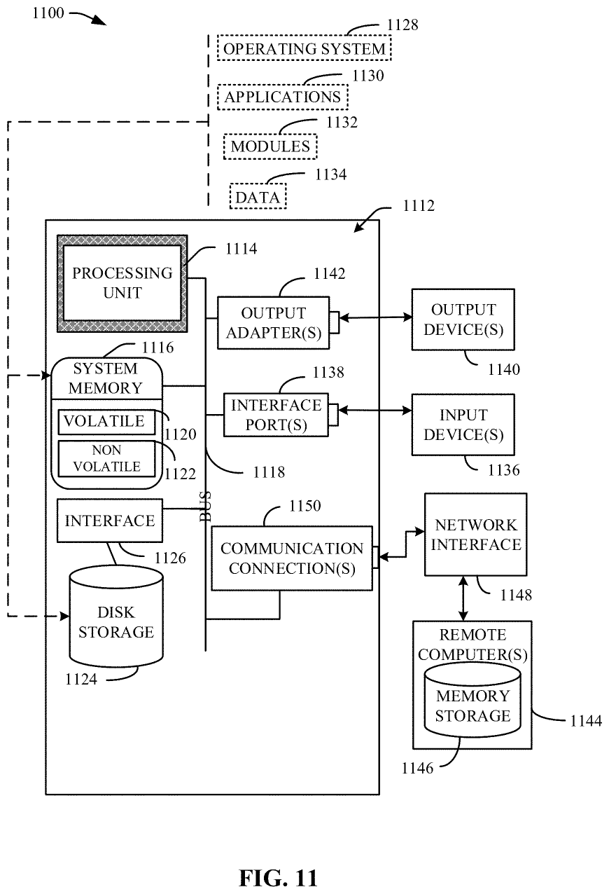

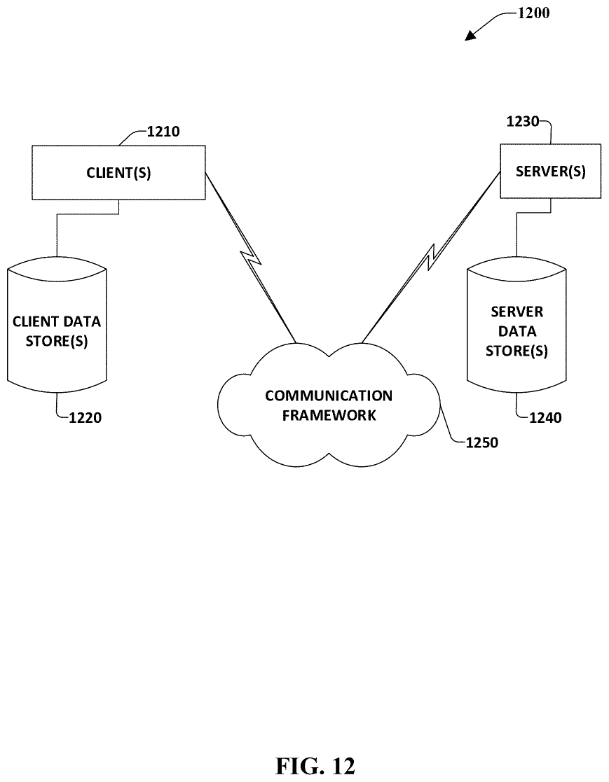

In order to provide a context for the various aspects of the disclosed subject matter, FIGS. 11 and 12 as well as the following discussion are intended to provide a brief, general description of a suitable environment in which the various aspects of the disclosed subject matter may be implemented.

With reference to FIG. 11, a suitable environment 1100 for implementing various aspects of this disclosure includes a computer 1112. The computer 1112 includes a processing unit 1114, a system memory 1116, and a system bus 1118. The system bus 1118 couples system components including, but not limited to, the system memory 1116 to the processing unit 1114. The processing unit 1114 can be any of various available processors. Dual microprocessors and other multiprocessor architectures also can be employed as the processing unit 1114.

The system bus 1118 can be any of several types of bus structure(s) including the memory bus or memory controller, a peripheral bus or external bus, and/or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 1394), and Small Computer Systems Interface (SCSI).

The system memory 1116 includes volatile memory 1120 and nonvolatile memory 1122. The basic input/output system (BIOS), containing the basic routines to transfer information between elements within the computer 1112, such as during start-up, is stored in nonvolatile memory 1122. By way of illustration, and not limitation, nonvolatile memory 1122 can include read only memory (ROM), programmable ROM (PROM), electrically programmable ROM (EPROM), electrically erasable programmable ROM (EEPROM), flash memory, or nonvolatile random access memory (RAM) (e.g., ferroelectric RAM (FeRAM). Volatile memory 1120 includes random access memory (RAM), which acts as external cache memory. By way of illustration and not limitation, RAM is available in many forms such as static RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM.

Computer 1112 also includes removable/non-removable, volatile/non-volatile computer storage media. FIG. 11 illustrates, for example, a disk storage 1124. Disk storage 1124 includes, but is not limited to, devices like a magnetic disk drive, floppy disk drive, tape drive, Jaz drive, Zip drive, LS-100 drive, flash memory card, or memory stick. The disk storage 1124 also can include storage media separately or in combination with other storage media including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage devices 1124 to the system bus 1118, a removable or non-removable interface is typically used, such as interface 1126.

FIG. 11 also depicts software that acts as an intermediary between users and the basic computer resources described in the suitable operating environment 1100. Such software includes, for example, an operating system 1128. Operating system 1128, which can be stored on disk storage 1124, acts to control and allocate resources of the computer system 1112. System applications 1130 take advantage of the management of resources by operating system 1128 through program modules 1132 and program data 1134, e.g., stored either in system memory 1116 or on disk storage 1124. It is to be appreciated that this disclosure can be implemented with various operating systems or combinations of operating systems.

A user enters commands or information into the computer 1112 through input device(s) 1136. Input devices 1136 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 1114 through the system bus 1118 via interface port(s) 1138. Interface port(s) 1138 include, for example, a serial port, a parallel port, a game port, and a universal serial bus (USB). Output device(s) 1140 use some of the same type of ports as input device(s) 1136. Thus, for example, a USB port may be used to provide input to computer 1112, and to output information from computer 1112 to an output device 1140. Output adapter 1142 is provided to illustrate that there are some output devices 1140 like monitors, speakers, and printers, among other output devices 1140, which require special adapters. The output adapters 1142 include, by way of illustration and not limitation, video and sound cards that provide a means of connection between the output device 1140 and the system bus 1118. It should be noted that other devices and/or systems of devices provide both input and output capabilities such as remote computer(s) 1144.

Computer 1112 can operate in a networked environment using logical connections to one or more remote computers, such as remote computer(s) 1144. The remote computer(s) 1144 can be a personal computer, a server, a router, a network PC, a workstation, a microprocessor based appliance, a peer device or other common network node and the like, and typically includes many or all of the elements described relative to computer 1112. For purposes of brevity, only a memory storage device 1146 is illustrated with remote computer(s) 1144. Remote computer(s) 1144 is logically connected to computer 1112 through a network interface 1148 and then physically connected via communication connection 1150. Network interface 1148 encompasses wire and/or wireless communication networks such as local-area networks (LAN), wide-area networks (WAN), cellular networks, etc. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 1150 refers to the hardware/software employed to connect the network interface 1148 to the bus 1118. While communication connection 1150 is shown for illustrative clarity inside computer 1112, it can also be external to computer 1112. The hardware/software necessary for connection to the network interface 1148 includes, for exemplary purposes only, internal and external technologies such as, modems including regular telephone grade modems, cable modems and DSL modems, ISDN adapters, and Ethernet cards.

FIG. 12 is a schematic block diagram of a sample computing environment 1200 with which the disclosed subject matter can interact. The sample computing environment 1200 includes one or more client(s) 1210. The client(s) 1210 can be hardware and/or software (e.g., threads, processes, computing devices). The sample computing environment 1200 also includes one or more server(s) 1230. The server(s) 1230 can also be hardware and/or software (e.g., threads, processes, computing devices). The servers 1230 can house threads to perform transformations by employing one or more embodiments as described herein, for example. One possible communication between a client 1210 and a server 1230 can be in the form of a data packet adapted to be transmitted between two or more computer processes. The sample computing environment 1200 includes a communication framework 1250 that can be employed to facilitate communications between the client(s) 1210 and the server(s) 1230. The client(s) 1210 are operably connected to one or more client data store(s) 1220 that can be employed to store information local to the client(s) 1210. Similarly, the server(s) 1230 are operably connected to one or more server data store(s) 1240 that can be employed to store information local to the servers 1230.

The present invention may be a system, a method, an apparatus and/or a computer program product at any possible technical detail level of integration. The computer program product can include a computer-readable storage medium (or media) having computer-readable program instructions thereon for causing a processor to carry out aspects of the present invention. The computer-readable storage medium can be a tangible device that can retain and store instructions for use by an instruction execution device. The computer-readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium can also include the following: a portable computer diskette, a hard disk, a random-access memory (RAM), a read-only memory (ROM), an erasable programmable read-only memory (EPROM or Flash memory), a static random-access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer-readable program instructions from the network and forwards the computer-readable program instructions for storage in a computer-readable storage medium within the respective computing/processing device. Computer-readable program instructions for carrying out operations of the present invention can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer-readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer-readable program instructions by utilizing state information of the computer-readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer-readable program instructions. These computer-readable program instructions can be provided to a processor of a general purpose computer, special-purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions can also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks. The computer-readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowcharts and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special-purpose hardware-based systems that perform the specified functions or acts or carry out combinations of special-purpose hardware and computer instructions.

While the subject matter has been described above in the general context of computer-executable instructions of a computer program product that runs on a computer and/or computers, those skilled in the art will recognize that this disclosure also can be implemented in combination with other program modules. Generally, program modules include routines, programs, components, data structures, etc. that perform particular tasks and/or implement particular abstract data types. Moreover, those skilled in the art will appreciate that the inventive computer-implemented methods can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments in which tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer-readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory. By way of illustration, and not limitation, nonvolatile memory can include read-only memory (ROM), programmable ROM (PROM), electrically programmable ROM (EPROM), electrically erasable PROM (EEPROM), flash memory, or nonvolatile random-access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed:

1. A computer-implemented system, comprising:
a memory; and
a processor that executes computer-executable components stored in the memory, the computer-executable components comprising:
an artificial intelligence (AI) component that assigns relevant medical categories, comprising at least type and context categories, to cataloged data from one or more medical reports, by incorporating a Semi Automated Curation (SAC) workflow;
an annotation component that generates AI based annotations for a cohort of medical reports of the one or more medical reports by incorporating an AI based Natural Language Processing (NLP) workflow;
a conversion component that converts, at least the AI based annotations, annotations generated by human entities, or a combination thereof, to standardized data formats; and
a machine learning component that uses completed annotations to train the AI based NLP workflow via neural network models, to enhance accuracy of the AI based NLP workflow.

2. The computer-implemented system of claim 1, wherein a data cataloging component catalogs the one or more medical reports to generate the cataloged data by incorporating an AI based data cataloging workflow.

3. The computer-implemented system of claim 1, wherein the AI component selects the AI based NLP workflow, from one or more existing AI based NLP workflows, based on assignment of the relevant medical categories.

4. The computer-implemented system of claim 1, wherein the AI component assists the human entities to create a new AI based NLP workflow.

5. The computer-implemented system of claim 1, wherein the standardized data formats comprise Fast Healthcare Interoperability Resources (FHIR) and Minimal common oncology data elements (Mcode) formats.

6. The computer-implemented system of claim 1, wherein the human entities at least accept, reject, rectify, or otherwise modify the AI based annotations to extract all potential annotations contained in the cohort of medical reports to generate the completed annotations.

7. The computer-implemented system of claim 1, further comprising:
an annotation dashboard component that displays, on an annotation dashboard, at least a completion status for annotations contained in the cohort of medical reports, the completion status generated by the AI component by incorporating an AI based completion workflow.

8. The computer-implemented system of claim 7, wherein the completion status corresponds to an amount of annotations extracted by human entities out of a total number of potential annotations detected by an annotation component, for a respective medical category.

9. The computer-implemented system of claim 1, wherein the AI component provides a report linking functionality such that upon selecting an entity on an annotation dashboard, a user of the annotation dashboard is redirected to a medical report of the cohort of medical reports that the entity is extracted from.

10. The computer-implemented system of claim 1, wherein the AI component further provides a relationship extraction functionality such that data from at least two or more medical reports of the cohort of medical reports is correlated and summarized.

11. The computer-implemented system of claim 1, wherein the AI component determines a degree of similarity between at least two or more medical reports of the cohort of medical reports, by incorporating an AI based similarity workflow, such that annotations are copied between the at least two or more medical reports based on the degree of similarity.

12. The computer-implemented system of claim 1, further comprising:
a data augmentation component that generates AI based synthetic data comprising additional annotated versions of information contained in the cohort of medical reports, to further assist the human entities to generate the completed annotations.

13. A computer-implemented method, comprising:
assigning, by a system operatively coupled to a processor, relevant medical categories, comprising at least type and context categories, to cataloged data from one or more medical reports, by incorporating an SAC workflow;
generating, by the system, AI based annotations for a cohort of medical reports of the one or more medical reports by incorporating an NLP based annotation workflow;
converting, by the system, at least the AI based annotations, annotations generated by human entities, or a combination thereof, to standardized data formats; and
training, by the system, the AI based NLP workflow via neural network models, using completed annotations, to enhance accuracy of the AI based NLP workflow.

14. The computer-implemented method of claim 13, further comprising:
cataloging, by the system, the one or more medical reports to generate the cataloged data by incorporating an AI based data cataloging workflow;
selecting, by the system, the AI based NLP workflow, from one or more existing AI based NLP workflows, based on assignment of the relevant medical categories; and
assisting, by the system, the human entities to create a new AI based NLP workflow.

15. The computer-implemented method of claim 13, wherein the standardized data formats comprise FHIR and Mcode formats.

16. The computer-implemented method of claim 13, further comprising:
displaying, by the system, at least a completion status for annotations contained in the cohort of medical reports, on an annotation dashboard, the completion status generated by an AI component by incorporating an AI based completion workflow.

17. The computer-implemented method of claim 13, further comprising:
providing, by the system, a report linking functionality such that upon selecting an entity on an annotation dashboard, a user of the annotation dashboard is redirected to a medical report of the cohort of medical reports that the entity is extracted from; and
providing, by the system, a relationship extraction functionality such that data from at least two or more medical reports of the cohort of medical reports is correlated and summarized.

18. The computer-implemented method of claim 13, further comprising:
determining, by the system, a degree of similarity between at least two or more medical reports of the cohort of medical reports, by incorporating an AI based similarity workflow, such that annotations are copied between the at least two or more medical reports based on the degree of similarity.

19. A computer program product comprising a non-transitory computer readable medium having program instructions embodied therewith, the program instructions executable by a processor to cause the processor to:
assign, by the processor, relevant medical categories, comprising at least type and context categories, to cataloged data from one or more medical reports, by incorporating an SAC workflow;
generate, by the processor, AI based annotations for a cohort of medical reports of the one or more medical reports by incorporating an NLP based annotation workflow;
convert, by the processor, at least the AI based annotations, annotations generated by human entities, or a combination thereof, to standardized data formats; and
training, by the processor, the AI based NLP workflow via neural network models, using completed annotations, to enhance accuracy of the AI based NLP workflow.

20. The computer program product of claim 19, wherein the program instructions are further executable by the processor to cause the processor to:
assist, by the processor, the human entities to create a new AI based NLP workflow;
display, by the processor, at least a completion status for annotations contained in the cohort of medical reports, on an annotation dashboard, the completion status generated by an AI component by incorporating an AI based completion workflow; and
determine, by the processor, a degree of similarity between at least two or more medical reports of the cohort of medical reports, by incorporating an AI based similarity workflow, such that annotations are copied between the at least two or more medical reports based on the degree of similarity.
